(19) United States
(12) Patent Application Publication
(10) Pub. No.: US 2019/0224104 A1
(43) Pub. Date: Jul. 25, 2019

(54) HAIR REPAIR COMPOSITION

(71) Applicant: RHODIA OPERATIONS, Paris (FR)

(72) Inventor: Katerina KARAGIANNI, Paris (FR)

(21) Appl. No.: 16/338,824

(22) PCT Filed: Oct. 3, 2017
(86) PCT No.: PCT/EP2017/075089

§ 371 Date: Apr. 2, 2019

(30) Foreign Application Priority Data
Oct. 4, 2016 (EP) 16192201.8

(51) Int. Cl. A61K 8/73; A61Q 5/00

(52) U.S. Cl. CPC A61K 8/737; A61Q 5/002

(57) ABSTRACT

The invention relates to a method for mending split ends of hair comprising contacting the hair with split ends with a composition comprising at least Cyamopsis tetragonoloba (guar) gum.

This application claims priority to European application No. 16192201.8 filed on Oct. 4, 2016, the whole content of this application being incorporated herein by reference for all purposes.

The present invention relates to hair care compositions for mending split ends, and more particularly to the use of Cyamopsis tetragonoloba (guar) gum as an agent for mending split ends.

Hair is a keratinous substance, which is repeatedly subjected to various stresses including especially environmental factors (such as exposure to UV radiation), damaging treatments (such as bleaching, coloring, perming or thermal straightening), and mechanical stressing, in particular during grooming procedures (for example by frequent brushing, back-combing or combing against high combing resistance).

It results in different types of structural damage to the hair: the cuticles are lifted, and the individual hair fibers may tend to become porous, to snarl, kink and/or interlock with each other.

Impact on the hair texture is noticeable for instance by a poor wet and dry combability, an increased electrostatic charge, increased brittleness, reduced maximum tear force and elongation at break of the hair and/or split ends, so that the hair as a whole looks unhealthy (dull, lifeless, . . . ), is hard to comb and/or feel coarse.

Among these damages, it is an object of the present invention to address the ever increasing demand in the market for hair care compositions which are useful for mending split ends.

The aim of the present invention is therefore to provide an ingredient which is useful for mending split ends.

“Split ends” refers to a condition wherein the ends of the hair are split into two or more shafts.

More especially it is defined as a longitudinal splitting of the hair fiber which develops after the protective cuticle has been stripped away from the end of the hair fibers as a result of either physical or chemical damaging of the hair. Split ends mainly form due to mechanical stresses during grooming routines and especially due to excessive combing forces.

Lubricating agents are already known to prevent or minimize formation of split ends. The lubrication reduces the friction in the hair during combing and hence reduces the strength of the abrasive forces to which the hair is being subjected. This is turn reduces the number of entanglements during the combing process.

The present invention, however, does not seek to prevent split end damages.

The present invention is concerned with split end mending, that is to say repair of existing damage by depositing substances that will restore axial cohesion to splits or “fill in” areas of shaft damage.

It is thus an object of the present invention to provide an ingredient which is effective in mending (i.e. repairing) split ends.

It is also an object of the present invention to provide an ingredient which is additionally effective in repairing hair cuticle damage and/or in aligning the hair fibers.

Repairing hair cuticle damage in the sense of the invention means smoothing hair cuticle. Visual effect may be observed for instance by looking at the hair fiber through scanning electron microscopy.

US 2005/0089494 and US 2006/0251603 disclose that the combination of polyquaternium-28 and methylvinylether/maleic acid copolymer, at particular ratios, produces a polyelectrolyte complex useful to repair split ends.

However, the stability of such polyelectrolyte complexes may be compromised when other ingredients are added to the hair care formulation. In particular, it has been reported that charged compounds and polymers could disrupt the structure of such polyelectrolyte complexes.

U.S. Pat. No. 6,258,348 discloses split-end mending compositions comprising three polymers: guar, a betaine-based polyurethane surfactant, and a silicone polyurethane. As demonstrated in EP1552807 the amphoteric or cationic guar gums disclosed in U.S. Pat. No. 6,258,348 do not achieve substantial split end repair in the absence of the other disclosed polymeric substituents.

The Applicant has now discovered unexpectedly that Cyamopsis tetragonoloba (guar) gum is useful as an agent for mending split ends.

There has been no suggestion in the prior art that Cyamopsis tetragonoloba (guar) gum, used alone, would make it possible to achieve substantial split end mending.

The subject of the invention is thus a method for mending split ends of hair comprising contacting the hair with split ends with a composition comprising at least Cyamopsis tetragonoloba (guar) gum.

The present invention is also directed toward a hair care composition for mending split ends comprising at least Cyamopsis tetragonoloba (guar) gum.

The present invention also relates to the use of at least Cyamopsis tetragonoloba (guar) gum, as an agent for mending split ends.

The present invention also relates to a method of mending split ends of hair comprising applying to the hair with split ends a composition comprising at least Cyamopsis tetragonoloba (guar) gum.

It has been found that Cyamopsis tetragonoloba (guar) gum of the invention not only provide a high percentage of split end mending, but are also able to close the split ends and to smooth the lifted cuticle scales so as to ensure a durable mend especially after combing or other stress factors during for example hair styling.

Advantageously, Cyamopsis tetragonoloba (guar) gum of the invention also provide split end mending and hair cuticle repair, without dry hair negatives such as the greasy appearance or feel, sticky feel, loss of gloss and/or heavy, coated feel that many consumers experience when conventional cationic polymers with high charge density and high molecular weight are used. According to anyone of the invention embodiments, a composition of the invention comprises from 0.01 to 2 pbw of Cyamopsis tetragonoloba (guar) gum relative to the total weight of the composition.

A composition of the invention may also comprise mixtures of Cyamopsis tetragonoloba (guar) gum with one or more different non-cellulosic polysaccharide derivative.

In one embodiment, the subject of the invention is a hair care composition for mending split ends comprising at least Cyamopsis tetragonoloba (guar) gum, and being devoid of any other ingredient acting as split end mending agent.

In other words, in one embodiment, a hair care composition for mending split ends of the invention comprises, as the sole agent for mending split ends, Cyamopsis tetragonoloba (guar) gum and contains no (0 pbw) other ingredient for that purpose.

Advantageously Cyamopsis tetragonoloba (guar) gum of the invention may be combined with a wide range of other hair benefit agents, including charged hair benefit agents. It is therefore possible to prepare hair care compositions for mending split ends including stable combinations of Cyamopsis tetragonoloba (guar) gum with other hair care ingredients that provide additional desirable properties.

Advantageously Cyamopsis tetragonoloba (guar) gum of the invention may be formulated in rinse-off or in leave-on hair care compositions. The performances in split end mending are satisfactory in both formulations.

According to anyone of the invention embodiments, the hair care composition comprising Cyamopsis tetragonoloba (guar) gum of the invention may be formulated as rinse-off or leave-on type of products.

As used herein, the expression “rinse-off compositions” means compositions which are rinsed off from the hair after application. Reversely the expression “leave-on compositions” means compositions which are not rinsed off from the hair after application.

The non-limiting examples of rinse-off products include shampoos, conditioners, hair straighteners, permanent waves, and hair colors (encompassing permanent, semi-permanent, and temporary hair colors).

Leave-on type of hair care products include but not limited to the following representative examples such as setting lotions, serums, hair sprays, mousses, hair lacquers, hair gels, hair waxes, styling creams, pomades, and tonics. Mention is made that the term “hair spray,” as used herein, refers to hair care products that are delivered in any atomized (spray) format, whether they be pressurized or unpressurized.

It is also considered to employ Cyamopsis tetragonoloba (guar) gum of the invention in the following non-limiting type of hair care and/or hair styling based end-user formulations such as 2 in 1 shampoos, leave-on and rinse-off conditioners, hair perming products, hair relaxants, permanent hair dyeing systems, hair styling mousses, semi-permanent hair dyeing systems, temporary hair dyeing systems, hair bleaching agents, permanent hair wave systems, hair setting formulations, non-coloring hair preparations, hair-frizz-control gels, hair leave-in conditioners, hair de-tangling products, hair fixatives, hair conditioning mists, hair care pump sprays and other non-aerosol sprays, hair cuticle coats.

Should the disclosure of any patents, patent applications, and publications which are incorporated herein by reference conflict with the description of the present application to the extent that it may render a term unclear, the present description shall take precedence.

The improved split ends mending achieved when using Cyamopsis tetragonoloba (guar) gum according to the invention can be emphasized on communication tools used by suppliers of chemical ingredients of hair care compositions, for example on animations or movies, presentations, leaflets, flyers, posters, technical data sheets, formularies, on any support, including on papers and websites. This can be linked to a complete or semi complete composition, or to a particular ingredient used to prepare a composition. The improved split ends mending achieved when using Cyamopsis tetragonoloba (guar) gum according to the invention can as well be emphasized on communication tools used in marketing hair care compositions, for example on commercial claims, labels, documentation linked to the composition, commercials, scientific studies backing commercial claims, on any support, including on papers, labels, websites, films or animation. Examples of commercial claims can include cure of split ends, deep repair of hair, split ends sealing or the like. Films or animation can for example show a hair fiber (or a representation thereof) having split ends and a product (or a representation thereof) approaching to the hair fiber and mending (i.e. repairing) split ends.

### EXAMPLES

The invention will now be described in further detail by way of the following non limiting examples, wherein the abbreviations have the usual meaning in the art. Water amount indicated as “q.s.” are intended to be “the amount required to complete to 100 pbw”.

All ingredients are expressed by weight percent of the whole formulation and as level of active ingredients.

The following serum compositions were prepared.

| | Control | | Comparative |
|---|---|---|---|
| | Formulation | Formulation 1 | Formulation |
| Cyamopsis | — | 1 | — |
| tetragonoloba | | | |
| (guar) gum (a) | | | |
| Jaguar C-13S (b) | — | — | 1 |
| Ethanol | 10 | 10 | 10 |
| Glycerin | 1 | 1 | 1 |
| Panthenol | 0.5 | 0.5 | 0.5 |
| Preservative | <0.5 | <0.5 | <0.5 |
| Water | q.s. | q.s. | q.s. |

(a) available from Rhodia
(b) available from Rhodia

### Formulation Procedure

Disperse the cationic guar derivative in water. Add glycerin and panthenol. Adjust pH to 4.5-5. Add the preservative and adjust the pH to 4,7. Add the ethanol and verify the pH (≤5). Add water to qsp 100.

### Assessment of the Performances on Hair Tresses

Hair tresses were purchased from Kerling International

European Natural Human Hair of the following characteristics: Color 5/0. 19 cm total length, 17 cm of free hair. 2.5 cm width. 4 g weight free hair.

### Hair Pre-Treatment Protocol

Wet the tress during 1 min under running water (or soak during 10 min in water if using several hair tresses). Apply on the whole tress 3 ml of a 10 pbw active sodium diethoxylated dodecyl sulfate (SLE2S) solution. Shampoo during 1 min, 30 sec on each side. Rinse during 1 min. Squeeze between index and middle finger. Detangle using middle teeth comb followed by fine teeth comb. Leave overnight in the controlled climate room (RH=50% ±10, T=23° C.).

### Hair Damaging Protocol to Obtain Split Ends

The repeated grooming apparatus, custom-built, is used. The device consists of 10 compartments and allows 10 hair tresses to be combed simultaneously. The 4 combs per compartment are mounted at 90° angles, allowing one complete revolution to comb the tress 3 times. Collection drawers are located under each tress to collect the broken fragments. All experiments were performed under controlled temperature and humidity conditions, in a climate controlled room (RH=50% ±10, T=23° C.).

The pre-treated hair tresses undergo repeated grooming during 7.5 hours at 19 rpm in order to create the split-ends.

### Measurement of the Percentage of Split End Repaired

The whole procedure takes place in the controlled climate room (RH=50% ±10, T=23° C.).

Use 4 g hair tresses pre-treated and then damaged according to the protocols described above. Pick 10 hair fibers all bearing split ends and glue them together with a scotch tape in order to obtain a 10-fiber kit. Count the split ends under the lighted magnifier. Weight 2 mg of the product (resp.: Formulation 1, Formulation 2 or Comparative Formulation) in a plastic weighting cup with the aid of a 0.5 ml capacity disposable transfer pipette dropper, on high precision balance. Gather the 10 fibers by holding them close to the split ends and soak them in the product to collect the maximum quantity. Spread the product out on the split ends (on the approximately last 1 cm towards the tips) with the finger until product absorption. Smooth the fibers once more and put the 10-fiber kit in the oven during 1 min. If needed smooth once to align the fibers and count the split ends left under the lighted magnifier.

The percent of split end repaired (% of Repair) was calculated as follows:

$$\% of Repair = \frac{( Original number of split ends ) - ( number of split ends left )}{Original number of split ends}$$

Results were the following:

| | Control | | Comparative |
|---|---|---|---|
| Formulation | Formulation | Formulation 1 | Formulation |
| % of Repair | 10 | 88 | 55 |
| (calculation) | | | |
| % of Repair | Reference | +++ | + |
| (trend) | | | |

Formulation 1, which include Cyamopsis tetragonoloba (guar) gum in accordance with the invention, exhibit significantly improved split end mending compared to the Comparative Formulation (which includes a non-cellulosic polysaccharide of the prior art outside the scope of the invention) and compared to the Control Formulation (which contains no ingredient acting specifically as agent for mending split ends).

Similar results were obtained using lower amounts of Cyamopsis tetragonoloba (guar) gum in accordance with the invention.

For instance, % of Repair higher than 90 were also achieved using 0.5 pbw of Cyamopsis tetragonoloba (guar) gum, formulated in the serum composition (i.e. control formulation) described previously.

These examples illustrate that Cyamopsis tetragonoloba (guar) gum according to the invention is highly effective in mending split ends.

1. A method for mending split ends of hair comprising contacting the hair with split ends with a composition comprising at least Cyamopsis tetragonoloba (guar) gum.

2. The method according to the preceding claims, wherein the composition comprises from 0.01 to 2 pbw of Cyamopsis tetragonoloba (guar) gum relative to the total weight of the composition.

3. A hair care composition for mending split ends comprising at least Cyamopsis tetragonoloba (guar) gum.

4. The composition according to claim 3, which is a leave-on composition.

5. The composition according to claim 3, which is a rinse-off composition.

6. Use of at least Cyamopsis tetragonoloba (guar) gum, as an agent for mending split ends.
